(19) United States
(12) Patent Application Publication
(10) Pub. No.: US 2004/0186428 A1
(43) Pub. Date: Sep. 23, 2004

(54) Auto-destructible syringe

(76) Inventor: Utpal Ray, Kolkata (IN)

Correspondence Address: Keith D. Nowak, DICKSTEIN SHAPIRO MORIN & OSHINSKY LLP, 41st Floor, 1177 Avenue of the Americas, New York, NY, 10036-2714, US

(21) Appl. No.: 10/658,373

(22) Filed: Sep. 10, 2003

Related U.S. Application Data
(60) Provisional application No. 60/409,901, filed on Sep. 11, 2002.

(51) Int. Cl. A61M 5/00

(52) U.S. Cl. 604/110

(57) ABSTRACT

The invention provides an auto-destructible syringe comprising a syringe barrel having an inner wall, a proximal end, and a distal end; a plunger slidably disposed within the syringe barrel; stopper means on the inner wall of the syringe barrel for preventing the syringe plunger from moving towards the distal end; grooves on the plunger alignable with the stopper means by axially rotating the plunger relative to the barrel; and cutting means connected to the plunger. When the stopper means are aligned with the grooves, the plunger can move toward the distal end of the syringe barrel. The cutting means cut the barrel when the plunger moves toward the distal end of the syringe barrel perforating the syringe barrel and preventing subsequent use of the syringe.

### CROSS-REFERENCE TO RELATED APPLICATION

This patent application claims the benefit of U.S. provisional patent application serial No. 60/409,901, filed Sep. 11, 2002, entitled AUTO-DESTRUCTIBLE, DISPOSABLE, BIODEGRADABLE SYRINGE. U.S. provisional patent application serial No. 60/409,901 is hereby incorporated by reference in its entirety.

### BACKGROUND OF THE INVENTION

Conventional disposable syringes available in the market are made of plastic material, which is non-biodegradable and dangerous for the environment. Used syringes may be contaminated with unused medications, pathogenic microorganisms, and other contaminants that pose a health risk to the public and the environment. Furthermore, these syringes are sometimes shared or even washed, repacked and placed back on the market, posing additional health hazards. Contaminated syringes lead to the spread of contagious diseases (e.g., AIDS and hepatitis). Therefore, it is desirable to render syringes inoperable after a single use to prevent re-use of the syringe.

Previously available syringes that are rendered inoperable after a single-use (“auto-destructible syringes”) have been described. U.S. Pat. No. 5,257,976 describes a single use disposable syringe that is destroyed upon its first use by a cutting element cutting the barrel. However, this syringe has no effective mechanism for preventing destruction by the cutting element during storage, filling, or any other mode. This can result in accidental destruction of the syringe prior to its initial use. Furthermore, the syringe is not biodegradable and requires advanced engineering methods, which increase the cost of the syringe.

U.S. Pat. No. 4,781,683 describes a method for providing a syringe that is rendered inoperative after a single injection. The syringe uses a hydrophilic expansion plug that is positioned in the fluid channel of the syringe. The hydrophilic expansion plug swells when exposed to water, blocking the fluid channel, and thereby preventing multiple use of the syringe. This method requires the absorption of some of the injection fluid, which typically is not desirable as it reduces the medicine content.

WO 97/26933 describes an auto-destruct disposable syringe which destroys the barrel of the syringe after the initial injection when attempting to refill the syringe. Thus, the syringe is not destroyed during the injection. This syringe also is not biodegradable.

What is needed is an auto-destructible syringe that is automatically destroyed during use and is resistant to premature destruction.

### BRIEF SUMMARY OF THE INVENTION

The invention provides auto-destructible, disposable syringes. The syringes are rendered inoperable after a single use, greatly reducing the health risks associated with the disposal of used syringes.

A preferred embodiment of the invention provides a syringe barrel having an inner wall, a proximal end, and a distal end; a plunger slidably disposed within the syringe barrel; stopper means on the inner wall of the syringe barrel for preventing the syringe plunger from moving towards the distal end; grooves on the plunger alignable with the stopper means by axially rotating the plunger relative to the barrel; and cutting means connected to the plunger. When the stopper means are aligned with the grooves, the plunger can move toward the distal end of the syringe barrel. The cutting means cut the barrel when the plunger moves toward the distal end of the syringe barrel.

The syringe barrel may be of any suitable shape (e.g., rectangular, cylindrical), length, and circumference for attachment to an injection device (e.g., needle, catheter). In one embodiment, the syringe barrel is made from a biodegradable material (e.g., compressed cardboard, paper, cellophane, glassine with gelatin, biodegradable waterproof coating, bamboo, and wood). In a preferred embodiment, the barrel is made of a cellulose derivative.

The syringe barrel may have an opening disposed longitudinally along the barrel. The opening may be of any suitable shape (e.g., rectangular, oval, triangular). The opening may be covered by a film which is cut by the cutting means to destroy the syringe. The film can be disposed on the inner wall of the syringe barrel. The film can be made from a biodegradable material (e.g., compressed cardboard, paper, cellophane, glassine with gelatin, biodegradable waterproof coating, bamboo, and wood) and is preferably transparent. The plunger may be made from a biodegradable material and is preferably made of a cellulose derivative. The syringe barrel can have a nozzle at one end configured to attach a needle to the syringe barrel.

The syringe barrel has a stopper means on the inner wall for preventing the plunger from moving towards the distal end when the stopper means is not aligned with the grooves in the plunger. The stopper means can be made from a biodegradable material (e.g., compressed cardboard, paper, cellophane, glassine with gelatin, biodegradable waterproof coating, bamboo, and wood). The size and shape of the stopper means corresponds with the size and shape of the groove in the plunger so the stopper means can engage the groove and permit the plunger to move downward toward the distal end of the syringe barrel.

The plunger is preferably made from a biodegradable material (e.g., compressed cardboard, paper, cellophane, glassine with gelatin, biodegradable waterproof coating, bamboo, and wood), has a groove or grooves for engaging the stopper means, and cutting means for cutting the syringe barrel or film.

The cutting means can be made from any material capable of cutting the syringe barrel or film (e.g., compressed cardboard, paper, bamboo, and wood). Alternatively, the cutting means comprise a material capable of perforating the film but incapable of perforating the syringe barrel. Preferably, the cutting means is made from sharpened bamboo, wood, or hardened cellulose. The cutting means is attached to the plunger and can perforate the syringe barrel or film when the plunger is moved distally. The cutting means can optionally be attached to the plunger by a resilient spring member (e.g., spring, wire) wherein the cutting means is retained by the inner wall of the syringe barrel and protrudes by its spring bias to perforate the film when the plunger grooves are aligned with the stopper means. .

Additional embodiments of the present invention are set forth in part in the description that follows, and in part will be obvious from the description, or may be learned through the practice of the invention. The objects and advantages of the invention will be attained by means of the instrumentalities and combinations particularly pointed out in the appended claims.

### BRIEF DESCRIPTION OF THE DRAWINGS

FIG. 1A shows the barrel of the syringe with biodegradable film and stopper means according to a preferred embodiment of the invention.

FIG. 1B shows a side view of the syringe barrel with the plunger, grooves, and cutting means according to a preferred embodiment of the invention.

FIG. 1C shows a bottom view of the plunger from the distal end of the syringe barrel after insertion into the syringe barrel according to an embodiment of the invention.

FIG. 1D shows a side view of the plunger with a groove according to an embodiment of the invention.

FIG. 1E shows a top view of the plunger with a groove aligned with the stopper means on the syringe barrel according to an embodiment of the invention.

FIG. 1F shows a side view of the plunger inserted into the syringe barrel with the groove and cutting means according to an embodiment of the invention.

### DETAILED DESCRIPTION OF THE INVENTION

Disposal of syringes poses significant health hazards for medical staff and the general public. According to the World Health Organization (WHO), approximately 12 billion injections are administered worldwide each year. World Health Organization. Vaccines, Immunization and Biologicals, Nov. 26, 2002. The majority of injections are therapeutic with immunization accounting for 5 to 10% of all injections. However, at least 30% of vaccine injections administered in developing countries are currently thought to be unsafe due to unsafe injection practices. Drain et al., Introducing auto-disable syringes into a developing country's immunization program, The 129th Annual Meeting of APHA (2001).

Unsafe injection practices result in millions of unnecessary cases of diseases such as hepatitis and AIDS. Safe Injection Globale Network (SIGN). Injection Safety. June 2002. Unsafe injection practices result in injuries to patients, transmission of disease to patients and medical workers, and disposal of hazardous waste in the community. Recycling syringes through repackaging and re-sale poses additional risks on an unsuspecting market place.

The present invention significantly reduces the risks associated with the re-use and disposal of syringes by providing a safe, environmentally-friendly, auto-destructible syringe. An embodiment of the invention comprises a syringe barrel having an inner wall, a proximal end, and a distal end; a plunger slidably disposed within the syringe barrel; stopper means on the inner wall of the syringe barrel for preventing the syringe plunger from moving towards the distal end; grooves on the plunger alignable with the stopper means by axially rotating the plunger relative to the barrel; and cutting means connected to the plunger. When the stopper means are aligned with the grooves, the plunger can move toward the distal end of the syringe barrel. The cutting means cut the barrel when the plunger moves toward the distal end of the syringe barrel.

The syringe barrel has a distal end and a proximal end. The distal end of the barrel may include a nozzle for fixing a needle or other injection device. The syringe barrel may be used to inject medications into humans, livestock, and other animals. The syringe barrel may have markings indicating the volume of fluid in the barrel. The syringe barrel may be configured in any suitable manner and shape for retaining and injecting medications. For example, the syringe barrel may be rectangular or cylindrical and may be of any length and circumference. The syringe barrel can be made from any suitable material and preferably is a biodegradable material (e.g., compressed cardboard, paper, cellophane, glassine with gelatin, biodegradable waterproof coating, bamboo, and wood). In a preferred embodiment, the barrel is made of a cellulose derivative. The syringe barrel and plunger or its extension can have markings to facilitate aligning the stopper means with plunger grooves. For example, the outer surface of the syringe barrel can have a marker indicating the location of the stopper means. The plunger can be rotated axially relative to the barrel until the plunger grooves are aligned with the marker indicating the location of the stopper means.

The syringe barrel can have an opening disposed longitudinally along the syringe barrel. The opening can optionally be covered by a film. In a preferred embodiment the relative tensile strength of the film is lower than the syringe barrel to facilitate destruction of the film by the cutting means. The film can be transparent to permit a user to observe the presence and/or volume of liquid contained within the barrel. Alternatively, the opening may be covered by a thinner portion of the same material comprising the inner wall of the syringe barrel. The thinner portion covering the opening is less resistant to cutting by the cutting means than the remaining portion of the syringe barrel.

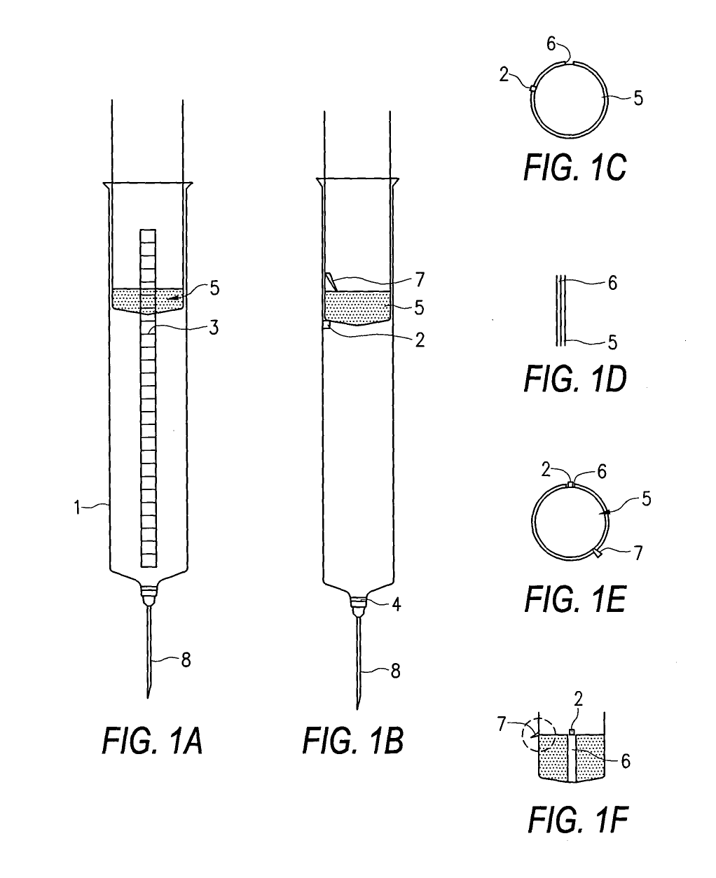

Referring to the drawings, FIG. 1A shows the syringe barrel (1), plunger (5), film (3), and needle (8). As shown in FIG. 1B, the stopper means (2) is disposed on the inner wall of syringe barrel (1) and prevents plunger (5) from moving downward . The distal end of syringe barrel (1) has nozzle (4) at its end for fixing needle (8). FIG. 1B shows a side view of plunger (5) inside syringe barrel (1). Cutting means (7) is shown attached to plunger (5).

FIG. 1C is a bottom view of the plunger in the syringe barrel. As shown in FIG. 1C, stopper means (2) is not aligned with groove (6)and therefore plunger (5) is prevented from moving downward toward the distal end of syringe barrel (1). As shown in FIGS. 1E and 1F, when plunger (5) is rotated axially so that groove (6) is aligned with stopper means (2), plunger (5) can be moved downward toward the distal end of the syringe barrel (1). As plunger (5) is moved downward toward the distal end of syringe barrel (1), cutting means (7) perforates syringe barrel (1) or optionally film (3) rendering the syringe inoperable for subsequent use.

Stopper means (2) prevents plunger (5) from moving towards the distal end of the syringe barrel when the plunger grooves are not aligned with the stopper means. Stopper means (2)is disposed on the inner wall of the syringe barrel (1). Liquid or another substance can be drawn into the syringe barrel by moving the plunger toward the proximate end of the syringe barrel. The stopper means prevents accidental or intentional movement of the plunger toward the distal end of the syringe barrel which may result in the unintentional destruction or perforation of the syringe prior to the first time use. The plunger grooves (6) are aligned with stopper means (2) by rotating the plunger axially relative to the barrel. When plunger grooves (6) are aligned with stopper means (2), the plunger can be moved distally.

Preferably, the stopper means is made from a biodegradable material (e.g., compressed cardboard, paper, cellophane, glassine with gelatin, biodegradable waterproof coating, bamboo, and wood). Stopper means (2) can be any suitable shape or size such that stopper means (2) can be inserted into groove (6) to permit movement of the plunger downward toward the distal end of the syringe barrel.

Plunger (5) is configured to be inserted into the syringe barrel. FIGS. 1A, 1B. For example, the outer diameter of the plunger may be slightly less than the inner diameter of the syringe barrel. Plunger (5) has one or more grooves (6) that can be aligned with stopper means (2) in order to permit movement of the plunger towards the distal end of the barrel. The plunger forms a seal with the inner wall of the syringe barrel to prevent leakage of medication or other fluids or substances during retraction of the plunger or during injection. The fit between plunger (5) and syringe barrel (1) should be sufficient to form a seal with the inner wall of the syringe barrel and permit axial rotation of plunger (5) to align stopper means (2) with grooves (6). The plunger can also have markings to facilitate aligning stopper means (2) with plunger grooves (6). For example, plunger (5) can have a marker on its proximal end indicating the location of plunger grooves (6). Plunger (5) can be rotated axially until the marker for grooves (6) is aligned with the marker for stopper means (2) on syringe barrel (1). The plunger can be made from biodegradable materials (e.g., compressed cardboard, paper, cellophane, glassine with gelatin, biodegradable waterproof coating, bamboo, and wood) and is preferably made from a cellulose derivative.

In one embodiment, the plunger further comprises an extension to facilitate rotating plunger (5) to align grooves (6) with stopper means (2). The plunger extension can be attached to the plunger and may have a handle at its proximal end disposed perpendicular to the axis of the plunger extension. The plunger extension may be knurled and/or covered with a material to facilitate gripping the plunger extension.

Cutting means (7) are attached to plunger (5) for perforating the syringe barrel rendering it inoperable for subsequent use. Cutting means (7) can be made from any material capable of perforating syringe barrel (1). Cutting means (7) can have a sharp edge and may be of any suitable shape and size capable of being attached to plunger (5) and perforating syringe barrel (1). Alternatively, cutting means (7) may be capable of perforating film (3) but not syringe barrel (1). In this embodiment, cutting means (7) only perforates film (3) when plunger (5) is moved distally, in that aligning the stopper means and plunger grooves also align the cutting means and the longitudinal opening in the barrel side wall which is covered by a film or other perforatable material. Cutting means (7) can be biodegradable. Preferably, the cutting means are made from sharpened bamboo, wood, or hardened cellulose.

In another embodiment, cutting means (7) comprises a resilient spring member that is biased against the inner wall. In this embodiment, cutting means (7) is retained by the inner wall of syringe barrel (1) and protrudes by the resilient spring member bias to perforate film (3) when plunger grooves (6) are aligned with stopper means (2).

The above description and appended drawings are only illustrative of embodiments which achieve the objects, features, and advantages of the present invention, and it is not intended that the present invention be limited thereto. Any modifications of the present invention which come within the spirit and scope of the following claims is considered part of the present invention.

What is claimed as new and desired to be protected by Letters Patent of the United States is:

1. An auto-destructible syringe comprising:
a syringe barrel having an inner wall, a proximal end, and a distal end;
a plunger slidably disposed within the syringe barrel;
stopper means on the inner wall of the syringe barrel for preventing the plunger from moving towards the distal end;
a groove on the plunger alignable with the stopper means by axially rotating the plunger relative to the barrel such that when the stopper means is aligned with the groove the plunger can move toward the distal end of the syringe barrel; and
cutting means connected to the plunger for cutting the syringe barrel when the plunger is moved toward the distal end of the syringe barrel.

2. The syringe of claim 1, wherein the syringe barrel is biodegradable.

3. The syringe of claim 1, wherein the syringe barrel comprises a material selected from the group consisting of compressed cardboard, paper, cellophane, gelatin, waterproof coating, bamboo, and wood.

4. The syringe of claim 1, wherein the syringe barrel further comprises a marking to facilitate aligning the groove with the stopper means.

5. The syringe of claim 1, wherein the plunger further comprises an extension to facilitate rotating the plunger to align the groove with the stopper means.

6. The syringe of claim 1, wherein the plunger comprises a biodegradable material.

7. The syringe of claim 1, wherein the plunger comprises a material selected from the group consisting of cellulose, compressed cardboard, paper, cellophane, glassine with gelatin, waterproof coating, bamboo, and wood rubber.

8. The syringe of claim 1, wherein the stopper means comprises a biodegradable material.

9. The syringe of claim 1, wherein the stopper means comprises a material selected from the group consisting of cellulose, compressed cardboard, paper, cellophane, glassine with gelatin, waterproof coating, bamboo, and wood rubber.

10. The syringe of claim 1, wherein the syringe barrel further comprises an opening disposed longitudinally along the barrel.

11. The syringe of claim 10, wherein the opening is covered by a film.

12. The syringe of claim 11, wherein the film is disposed on the inner wall of the barrel.

13. The syringe of claim 10, wherein the cutting means is aligned with the opening when the stopper means is aligned with the plunger groove.

14. The syringe of claim 11, wherein the film comprises a biodegradable material.

15. The syringe of claim 1, wherein the cutting means comprises biodegradable material.

16. The syringe of claim 1, wherein the cutting means comprises a material selected from the group consisting of bamboo, wood, and hardened cellulose.

17. The syringe of claim 11, wherein the cutting means comprises a material capable of perforating the film but incapable of perforating the syringe barrel.

18. The syringe of claim 11, wherein the cutting means comprises a resilient spring member that is biased against the inner wall.

19. The syringe of claim 18, wherein the cutting means is retained by the inner wall of the syringe barrel and protrudes outwardly by its spring bias to perforate the film when the plunger grooves are aligned with the stopper means.
